(19) United States
(12) Patent Application Publication
(10) Pub. No.: US 2005/0026878 A1
(43) Pub. Date: Feb. 3, 2005

(54) Steroid modulators in the treatment of peripheral nerve sheath tumors

(76) Inventors: Margaret McLaughlin, Cambridge, MA (US); Tyler Jacks, West Newton, MA (US)

Correspondence Address: CLARK & ELBING LLP, 101 FEDERAL STREET, BOSTON, MA, 02110, US

(21) Appl. No.: 10/626,312

(22) Filed: Jul. 24, 2003

Related U.S. Application Data
(60) Provisional application No. 60/398,647, filed on Jul. 25, 2002.

(51) Int. Cl. A61K 31/56

(52) U.S. Cl. 514/169; 514/182

(57) ABSTRACT

The invention features methods for treating and preventing a peripheral nerve sheath tumor in a mammal such as a human. The methods involve administering to the mammal a compound that modulates the biological activity of a gonatropic steroid receptor, such as the progesterone receptor in an amount sufficient to inhibit the growth of the peripheral nerve sheath tumor. The mammal may be administered with the compound alone or in combination with a second therapeutic regimen. Also disclosed are screening methods that make use of gonatropic steroid receptors for the identification of novel therapeutics for PNSTs.

### CROSS REFERENCE TO RELATED APPLICATIONS

This application claims benefit from U.S. Ser. No. 60/398,647, filed Jul. 25, 2002, now pending.

### STATEMENT AS TO FEDERALLY SPONSORED RESEARCH

The present research was supported by a grant from the United States Army Neurofibromatosis Research Program (DAMD 17-00-1-0594). The U.S. government has certain rights to this invention.

### FIELD OF THE INVENTION

The present invention relates to the treatment or prevention of peripheral nerve sheath tumors.

### BACKGROUND OF THE INVENTION

Tumors derived from cells surrounding the axons of peripheral nerves are collectively termed peripheral nerve sheath tumors (PNSTs), and are among the most common forms of clinically diagnosed benign tumors. The majority of tumors resulting from the neural sheath are neurofibromas and schwannomas, accounting for 66% and 20% of PNSTs respectively. PNSTs afflict individuals, showing no bias towards gender or ethnicity. While the majority of PNSTs are benign, malignancy may arise from neurofibromas, which are then referred to as malignant peripheral nerve sheath tumors (MPNST). MPNSTs may account for 10% of all soft tissue sarcomas.

Approximately one third of all patients diagnosed with neurofibromas, the most common PNST, have been clinically diagnosed with type-I neurofibromatosis (NF-1 or von Recklinghausin's disease) while the other two thirds of neurofibromas have arisen by sporadic mutations. Type-I neurofibromatosis is a genetic disorder, giving rise to neurofibromas and may produce abnormalities such as skin changes and bone deformities.

Type-I neurofibromatosis is an autosomal dominant genetic disorder that has been linked to the NF1 gene. NF-1 or von Recklinghausin's disease affects 1:2500-3500 individuals. The NF1 gene is located on the long arm of human chromosome 17 (17q 11.2) and encodes a tumor suppressor protein, neurofibromin. Studies have shown that loss of neurofibromin expression results in NF-1. Further clinical studies demonstrated that while the severity of neurofibromas in NF-1 patients is variable, the penetrance is virtually 100%. NF1 mutations also account for 40-60% of all malignant PNSTs. Patients with NF-1 are observed to be predisposed to myelogenous leukemias, pheochromocytomas, rhabdomyosarcomas, pilocytic astrocytomas, and gastrointestinal stromal tumors.

Currently, surgical resection is the only means of treating neurofibromas. However, complete surgical resection is not always possible for large lesions or for NF-1 patients who may have hundreds to thousands of lesions. Furthermore, surgical resection of intraneural neurofibromas (localized intraneural, plexiform, and massive soft tissue subtypes) requires sacrifice of the parent nerve leading to significant neurological deficits. Malignant transformation to MPNSTs occurs in a significant fraction of plexiform neurofibromas. Treatment for MPNSTs consists of surgical resection followed by radiation therapy. Even with aggressive treatment the prognosis is poor with 10-year survival rates ranging from 23-34%.

Thus, a non-surgical treatment for PNSTs would be highly desirable.

### SUMMARY OF THE INVENTION

The present invention features methods for the prophylaxis and the treatment of peripheral nerve sheath tumors. This method involves administering to a mammal in need thereof a therapeutically effective dose of a compound that modulates the biological activity of a gonadotropic steroid receptor (e.g., progesterone receptor, estrogen receptor, androgen receptor). This invention is based on our discovery that the progesterone receptor (PR) is expressed in a high percentage of human neurofibromas, in a manner consistent with the involvement of PRs in the development of peripheral nerve sheath tumors (PNSTs). Based on our results, gonatropic steroid receptors, such as the PR ligand progesterone, play an important role in tumor growth and therefore, modulation of the PR activity can successfully treat PNSTs.

In a first aspect, the invention features a method for treating or preventing a peripheral nerve sheath tumor by administering to a mammal a therapeutically effective dose of a compound that modulates the biological activity of gonatropic steroid receptor (e.g., progesterone receptor, estrogen receptor, or androgen receptor). The compound of the invention may be any therapeutic agent that modulates the biological activity of a gonatropic steroid receptor, such as a neutralizing antibody, a small molecule agonist, a small molecule antagonist, an antisense molecule, or a double-stranded interference RNA (RNAi).

In all foregoing aspects of this invention, the mammal being treated is preferably a human. Peripheral nerve sheath tumors being treated include, for example, neurofibromas, schwannomas, perineuriomas, malignant peripheral nerve sheath tumors, and Triton tumors. The neurofibroma may be sporadic. The neurofibroma may also be associated with type-I neurofibromatosis. If desired, the mammal being treated may also receive a second therapeutic regimen (e.g., tumor resection, chemotherapy, or radiotherapy).

In another related aspect, the invention features a method of treating or preventing a peripheral nerve sheath tumor. This method includes administering to a mammal in need thereof a therapeutically effective dose of a combination of compounds that modulates the biological activity of more than one gonatropic steroid receptors, such as the progesterone, estrogen, or androgen receptor. Desirably, the combination of compounds modulates the biological activity of any two of the progesterone, estrogen, or androgen receptor. More desirably at least one compound in the combination includes a modulator of the biological activity progesterone receptor.

In another related aspect, the invention features a method for monitoring the progression of a peripheral nerve sheath tumor. This method includes the step of measuring the amount of a gonadotropic steroid receptor MRNA or polypeptide expression in a sample from a subject, an increase or decrease in the gonadotropic steroid receptor mRNA or polypeptide expression in the sample, relative to a control sample, indicating a progression of a peripheral nerve sheath tumor or a propensity thereto in the subject.

In a related aspect, the invention features a method for determining a course of treatment for a mammal diagnosed as having a peripheral nerve sheath tumor. This method includes the step of (a) providing a histological preparation of a peripheral nerve sheath tumor from the mammal, and (b) detecting the presence of a gonadotropic steroid receptor in the histological preparation. The presence of the gonadotropic steroid receptor identifies the mammal as being a candidate for treatment with a compound that modulates the biological activity of a gonatropic steroid receptor. The compound may inhibit or activate the gonatropic steroid receptor. Preferably, the gonatropic receptor being detected is the progesterone receptor.

In another related aspect, the invention features a method for treating a peripheral nerve sheath tumor. This method includes the step of introducing a progesterone, estrogen, or androgen receptor antisense nucleic acid that inhibits progesterone, estrogen, or androgen receptor biological activity, regardless of length of the antisense nucleic acid.

In yet another related aspect, the invention features a method for treating a peripheral nerve sheath tumor. This method includes the step of introducing a transgene encoding a progesterone, estrogen, or androgen receptor polypeptide. The transgene being operably linked to expression control sequences, and the transgene being positioned for expression in peripheral nerve sheath tumor cells.

The transgene can encode a wildtype or dominant negative variant of the progesterone, estrogen, or androgen receptor.

In yet another related aspect, the invention features a method for treating a peripheral nerve sheath tumor. This method includes the step of introducing a progesterone, estrogen, or androgen receptor double-stranded interference ribonucleic acid (RNAi) that inhibits progesterone, estrogen, or androgen receptor biological activity, regardless of length of the RNAi nucleic acid.

In an embodiment to the previous three aspects, the receptor is the progesterone receptor.

In another aspect, the invention provides a method for identifying a candidate compound for treating or preventing a PNST in a mammal. The method involves the steps of: (a) contacting a cell expressing a gonatropic steroid receptor gene with a candidate compound; and (b) measuring gonatropic steroid receptor gene expression or gonatropic steroid receptor protein activity in the cell. A candidate compound that modulates the expression or the activity of gonatropic steroid receptor, relative to gonatropic steroid receptor expression or activity in a cell not contacted with the candidate compound, is identified as a candidate compound useful for treating, or preventing PNSTs in a mammal.

In preferred embodiments, the gonatropic receptor gene is a gonatropic steroid receptor fusion gene and the gonatropic receptor-expressing cell is a mammalian cell (e.g., a rodent cell). In other embodiments, step (b) involves the measurement of gonatropic receptor MRNA or protein.

In a related aspect, the invention provides another method for identifying a candidate compound for treating or preventing a PNSTin a mammal. This method involves the steps of: (a) contacting a gonatropic steroid receptor protein with a candidate compound; and (b) determining whether the candidate compound binds the gonatropic steroid receptor protein and/or modulates gonatropic steroid receptor activity. Candidate compounds that bind and modulate gonatropic steroid receptor activity are identified as candidate compounds useful for treating or preventing a PNST, in a mammal.

In preferred embodiments, the method also tests the ability of the candidate compound to modulate the expression of the gonatropic receptor gene in a cell, for example a mammalian cell such as a rodent or human cell. Most preferably, the gonatropic receptor is a human gonatropic receptor.

In yet another related aspect, the invention features a method for identifying combinations of compounds useful for treating or preventing a PNST in a mammal. This method includes the steps of (a) contacting a cell expressing a gonatropic steroid receptor gene with a combination of agents, including (i) mifepristone and (ii) a candidate compound, and (b) measuring the gene expression or protein activity the gonatropic steroid receptor in the cell, such that the combination that modulates the expression or activity of the gonatropic steroid receptor relative to a cell contacted with only mifepristone or the candidate compound, is identified as a combination useful for the treatment and prevention of a PNST.

In yet another related aspect, the invention features a method for identifying combinations of compounds useful for treating a patient having a peripheral nerve sheath tumor. This method includes the steps of (a) contacting peripheral nerve sheath tumor cells in vitro with (i) mifepristone and (ii) a candidate compound, and (b) determining whether the combination of the mifepristone and the candidate compound reduces the growth of the peripheral nerve sheath tumor cells relative to peripheral nerve sheath tumor cells contacted with the mifepristone but not contacted with the candidate compound, or peripheral nerve sheath tumor cells contacted with the candidate compound but not with the mifepristone. A reduction in growth identifies the combination as a combination that is useful for treating a patient having a peripheral nerve sheath tumor.

In an embodiment to the previous aspect, the peripheral nerve sheath tumor is selected from a group consisting of neurofibromas, schwannomas, perineuriomas, malignant peripheral nerve sheath tumors, and Triton tumors. Desirably, the peripheral nerve sheath tumor is a neurofibroma. More desirably, the neurofibroma is a sporadic neurofibroma or the neurofibroma is associated with type-1 neurofibromatosis.

A progesterone receptor of the invention can be either the progesterone receptor A isoform, or progesterone receptor B isoform. The progesterone receptor can exist in a monomeric or dimeric form. The dimeric form can be a homodimer or a heterodimer of the PR-A and PR-B isoforms.

As used herein, by “peripheral nerve sheath tumors” or “PNSTs” is meant any tumors of the nerve sheath (the cells surrounding the axons of peripheral nerves), whether benign or malignant. The principal cells of the nerve sheath are Schwann cells, perineurial cells, and fibroblast-like cells. These tumors include, for example, neurofibromas, schwannomas, perineuriomas, ossifying, malignant peripheral sheath tumors, and Triton tumors.

Neurofibromas vary in size and severity. They are more likely to cause pain than schwannomas. Neurofibromas can be solitary or plexiform (a weblike network or interjoining network that runs along the nerve) which may grow along the length of the nerve, thereby compressing it. Roughly two-thirds of neurofibromas arise sporadically while one-third are in association with type-1 neurofibromatosis (NF-1).

NF-1 is a complex neurocutaneous disorder in which patients suffer from multiple lesions of diverse type (hyperplasias, hypoplasias, hamartomas, and neoplasms), indicating that the NF1 gene product has dual functions in development and cell cycle control. The gene responsible for NF-1 maps to the pericentric region of chromosome 17 q and encodes the protein neurofibromin. Neurofibromin is expressed ubiquitously but is most abundant in the central and peripheral nervous systems. Neurofibromin is a member of the Ras-specific GTPase activating protein (RasGAP) family and accelerates the conversion of active GTP-bound Ras to inactive GDP-bound Ras, thereby leading to a decrease in cell proliferation and/or cell survival.

Neurofibromas invariably contain a variety of cell types found in normal peripheral nerves, including axonal processes of neurons, Schwann cells, perineurial cells, fibroblasts, and mast cells. Recent studies using cultured Schwann cells from human neurofibromas and from conditional Nf1 mouse models have clearly demonstrated that in NF-1 patients and in mice with genetically engineered Nf1 mutations, a complete loss of the NF1 gene occurs within Schwann cells, confirming that the Schwann cell is the cell of origin of the tumor.

The cellular heterogeneity of neurofibromas raises the question of what role the non-neoplastic tumor-associated cells play in tumor growth. Reciprocal signaling is known to occur among the various cell types within peripheral nerves and likely occurs between cells in a neurofibroma. Even partial reductions in neurofibromin levels may result in inappropriate responses to such signals. Defects in several neurofibroma-associated Nf1± cell types have been observed. In cellular studies Nf1± Schwann cells are able to induce angiogenesis and are more invasive than their wild-type counterparts. Embryonic fibroblasts and mast cells derived from Nf1± mice hyperproliferate in vivo and in vitro. Most intriguing is the observation that Nf1 heterozygosity in the host environment promotes the growth of neurofibromas in conditional Nf1mice

By “compound” is meant any therapeutic agent that can modulate (e.g., increase or decrease) the biological activity of a gonatropic steroid receptor. Such compounds include, for example, small molecule antagonists or agonists, neutralizing antibodies, dominant negative molecules, antisense nucleic acids, and interference RNA (RNAi) nucleic acids.

By “antisense,” as used herein in reference to nucleic acids, is meant a nucleic acid sequence, regardless of length, that is complementary to the coding strand or mRNA of a gonadotropic steroid receptor gene. Desirably the antisense nucleic acid is capable of decreasing the expression of a gonadotropic steroid receptor in a cell. Desirably the decrease is relative to a control, 90%, more desirably 75%, and most desirably 50% or more. Thus, the amount of reduction may be 10%, 20%, 25%, 30%, 40%, 50%, 60%, 70%, 75% 80%, 90% or even 100% relative to a control. Desirably a gonadotropic steroid receptor antisense nucleic acid includes from about 8 to 30 nucleotides. A gonadotropic steroid receptor antisense nucleic acid may also contain at least 40, 60, 85, 120, or more consecutive nucleotides that are complementary to a gonadotropic steroid receptor mRNA or DNA, and may be as long as a full-length gonadotropic steroid receptor gene or mRNA. The antisense nucleic acid may contain a modified backbone, for example, phosphorothioate, phosphorodithioate, or other modified backbones known in the art, or may contain non-natural intemucleoside linkages.

By “positioned for expression” is meant that the DNA molecule is positioned adjacent to a DNA sequence, which directs transcription and translation of the sequence (i.e., facilitates the production of, e.g., a gonadotropic steroid receptor polypeptide)

By “operably linked” is meant that a nucleic acid molecule and one or more regulatory sequences (e.g., a promoter) are connected in such a way as to permit expression and/or secretion of the product (i.e., a polypeptide) of the nucleic acid molecule when the appropriate molecules (e.g., transcriptional activator proteins) are bound to the regulatory sequences.

By “differentially expressed” refers to a difference in the expression level of a nucleic acid. This difference may be either an increase or a decrease in expression, when compared to control conditions.

By “cancer,” “neoplasia,” “neoplasm,” or “tumor” is meant a cell or tissue multiplying or growing in an abnormal manner. Cancer growth is uncontrolled and progressive, may be benign or malignant, and occurs under conditions that would not elicit, or would cause cessation of, multiplication of normal cells.

By “detecting” is meant any means for marking or identifying the presence of a molecule, for example, a polypeptide or fragment thereof, or a nucleic acid molecule. Methods for detecting a molecule are well known in the art and include, without limitation, reverse transcriptase-mediated polymerase chain reaction (RT-PCR), Northern blotting, RNase protection assay, immunohistochemical staining, immunoblotting, immunoprecipitation, and mass spectroscopy.

By “assaying” is meant analyzing the effect of a treatment, be it chemical or physical, administered to whole animals or cells derived therefrom. The material being analyzed may be an animal, a cell, a lysate or extract derived from a cell, or a molecule derived from a cell. The analysis may be, for example, for the purpose of detecting altered gene expression, altered RNA stability, altered protein stability, altered protein levels, or altered protein biological activity. The means for analyzing may include, for example, antibody labeling, immunoprecipitation, phosphorylation assays, and methods known to those skilled in the art for detecting nucleic acids and polypeptides.

By “gonadotropic steroid receptor” is meant a family of nuclear hormone receptors capable of specific binding to the sex hormones, androgens, progestins, and estrogens. These nuclear hormone receptors are ligand-inducible transcription factors. Each sex hormone has a corresponding nuclear hormone receptor, for instance, progestins bind to the progesterone receptor, NR3C3; the cognate receptor for androgens is the androgen receptor, NR3C4; and estrogens are associated with the estrogen receptors, NR3A1 and NR3A2. The human progesterone receptor polypeptide and nucleotide sequence may be substantially identical to GenBank Accession Number NM_{—}00926; an androgen receptor polypeptide and nucleotide sequence may be substantially identical to GenBank Accession Numbers NM_{—}000044 and XM_{—}010429; and an estrogen receptor polypeptide and nucleotide sequence may be substantially identical to GenBank Accession Numbers XM_{—}045967 and NM_{—}000125, or to NM_{—}001437 and AF051427.

Some of the known biological activities of the gonadotropic steroid receptors can be described through shared common functional domains, designated A through F. The amino terminal A/B region has variable length among members of the steroid receptor superfamily, and encodes a transcriptional activation domain, termed AF-1. The C domain comprises two zinc-finger-like motifs, involved in DNA binding. A variable length hinge region defines domain D. The E region encompasses the ligand specificity domain, and termed the ligand-binding domain (LBD). A second activation domain (AF-2), a dimerization domain, and a region involved in nuclear localization also reside in the E region. The most carboxy terminal region, F, has yet to be ascribed a function, and in some members of the steroid receptor superfamily (e.g. the progesterone receptor), is absent.

The biological activities of the gonadotropic steroid receptors include nuclear translocation mediated by a nuclear localization signal, transactivation or transrepression of target genes; DNA binding activity; the ability to interact with transcriptional coregulators such as steroid receptor coactivator-1 (SRC-1), GRIP 1/TIF2, P/CAF, CBP/p300, and Rap46. A more complete list of co-regulator interactions and known biological activities of the gonadotropic steroid receptors can be found in McKenna and Malley, Cell 108:465-474, 2002 and in Robyr et al., Mol Endo 14:329-347, 2000 and herein incorporated by reference.

By “dimer” is meant a protein-protein interaction composed a gonadotropic receptor monomer with a transcriptional binding partner. For example, PR-A can dimerize with PR-B. In most contexts PR-B acts as a transcriptional activator, whereas PR-A acts as a transcriptional repressor. PR-A is a transcriptional repressor of PR-B as well as of estrogen, glucocorticoid, androgen, and mineralcorticoid receptors. PR-A can heterodimerze with PR-B, and may inhibit PR-B directly. PR-A, however, cannot heterodimerize with ER, suggesting that the mechanism of transcriptional interference is indirect and may involve binding to corepressors. Further guidance for assaying protein interactions or function may be found in, for example, Ausubel et al. (Current Protocols in Molecular Biology, John Wiley & Sons, New York, 2000).

By “substantially identical” is meant an amino acid sequence which differs only by conservative amino acid substitutions, for example, substitution of one amino acid for another of the same class (e.g., valine for glycine, arginine for lysine, etc.) or by one or more non-conservative substitutions, deletions, or insertions located at positions of the amino acid sequence which do not destroy the function of the protein or domain (assayed, e.g., as described herein). Desirably, the amino acid sequence is at least 60%, desirably, 70%, more desirably, 85%, and, most desirably, 95% identical to the sequences of the aforementioned progesterone, estrogen, or androgen receptors. A “substantially identical” nucleic acid sequence codes for a substantially identical amino acid sequence as defined above.

By “modulate” is meant conferring a change, either by a decrease or increase in gonadotropic steroid receptor protein, MRNA or gonadotropic steroid receptor biological activity that is naturally present within a particular cell or sample. Desirably, the change in response is at least 5%, more desirably, the change in response is 20% and most desirably, the change in response level is a change of more than 50% relative to the levels observed in naturally occurring gonadotropic steroid receptor biological activity.

By “pharmaceutically acceptable carrier” is meant a carrier that is physiologically acceptable to the treated mammal while retaining the therapeutic properties of the compound with which it is administered. One exemplary pharmaceutically acceptable carrier is physiological saline. Other physiologically acceptable carriers and their formulations are known to one skilled in the art and described, for example, in “Remington: The Science and Practice of Pharmacy” (20th ed., ed. A. R. Gennaro AR., 2000, Lippincott Williams & Wilkins)

By “protein” or “polypeptide” is meant any chain of amino acids, regardless of length or post-translational modification (for example, glycosylation or phosphorylation).

By “RNAᵢ” or “RNA interference,” as used herein in reference to nucleic acids, is meant a ribonucleic acid sequence, regardless of length, that is double-stranded to the coding or non-coding strand of the mRNA of a gonadotropic steroid receptor gene. Double-stranded RNA (dsRNA) directs gene-specific, post-transcriptional silencing in many organisms, including vertebrates. Preferably, the RNAᵢ is capable of decreasing the expression of a gonadotropic steroid receptor in a cell. Desirably the decrease is relative to a control, 90%, more desirably 75%, and most desirably 50% or more. Desirably a gonadotropic steroid receptor RNAᵢ includes from about 20 to 30 nucleotides. A gonadotropic steroid receptor RNAᵢ may also contain at least 40, 60, 85, 120, or more consecutive nucleotides that are complementary to a gonadotropic steroid receptor mRNA or DNA, and may be as long as a full-length gonadotropic steroid receptor gene or mRNA. The RNAᵢ nucleic acid may contain a modified backbone, for example, phosphorothioate, phosphorodithioate, or other modified backbones known in the art, or may contain non-natural internucleoside linkages. Methods of use of RNAᵢ are known to those skilled in the art and can found, for example, in, Zamore et al., 2000, Cell 101:25-33 or Tuschl et al., 1999, Genes Dev 13:3191-3197, and herein incorporated by reference.

By a “therapeutically effective amount” is meant an amount sufficient to result in the inhibition of peripheral nerve sheath tumor progression. It will be appreciated that there will be many ways known in the art to determine the therapeutic amount for a given application. For example, the pharmacological methods for dosage determination may be used in the therapeutic context.

By “treating or preventing a PNST” is meant preventing, reducing, or eliminating the tumor in a subject before or after it has occurred. As compared with an equivalent untreated control, such reduction or degree of prevention is at least 5%, 10%, 20%, 40%, 50%, 60%, 80%, 90%, 95%, or 100% as measured by any standard technique known in the art.

By a “candidate compound” is meant a chemical, be it naturally-occurring or artificially-derived, that is assayed for its ability to modulate an alteration in reporter gene activity or protein levels, by employing one of the assay methods described herein. Test compounds may include, for example, peptides, polypeptides, synthesized organic molecules, naturally occurring organic molecules, nucleic acid molecules, and components thereof.

### BRIEF DESCRIPTION OF THE DRAWINGS

FIGS. 1A-1D are photographs of immunohistochemical preparations of human peripheral nerve tissue and probed for progesterone receptor expression. Immunohistochemistry with an anti-PR antibody demonstrates that scattered cells within neurofibromas have nuclear staining for PR (brown) (FIG. 1B). Schwannomas (FIG. 1C) and malignant peripheral nerve sheath tumors (FIG. 1D) as well as normal peripheral nerve (FIG. 1A), relative to neurofibromas, score less for PR expression (Scale bars=50μm).

FIGS. 2A and 2B are photographs of immunohistochemical preparations of human peripheral nerve tissue and probed for progesterone receptor expression. Isoforms A and B of PR are expressed in human neurofibromas, as demonstrated by staining with antibodies specific for isoform A of PR (FIG. 2A) or isoform B of PR (FIG. 2B). Results demonstrate that scattered cells within neurofibromas have nuclear staining for both isoforms (Scale bars=10μm).

FIGS. 3A-3F are photographs of immunofluoresence preparations of human peripheral nerve tissue and probed for progesterone receptor expression. Neoplastic Schwann cells within neurofibromas do not express PR. PR immunofluoresence alone (FIG. 3A). Neurofibromin immunofluoresence alone (FIG. 3B). Overlay of PR and neurofibromin immunofluoresence showing the presence of cells expressing both PR and neurofibromin (FIG. 3A). PR immunofluoresence alone (FIG. 3D). S-100 immunofluoresence alone (FIG. 3E). Overlay of PR and S-100 immunofluoresence demonstrating that PR and S-100 do not colocalize (FIG. 3A) (Scale bars=10μm).

FIG. 4 is a photograph of an immunohistochemical preparation of human peripheral nerve tissue and probed for estrogen receptor (ER) expression. ER is expressed in a minority of neurofibromas. Immunohistochemistry with an anti-ER antibody demonstrates that scattered cells within neurofibromas have nuclear staining for ER (Scale bar=50μm).

### DETAILED DESCRIPTION

In general, the present invention features methods for the treatment and prophylaxis of peripheral nerve sheath tumors (PNSTs) by modulating the biological activity of gonatropic steroid receptors, such as the progesterone receptor, androgen receptor, and estrogen receptor. Also disclosed are screening methods for the identification of candidate compounds useful for the treatment and prevention of PNSTs. These screening methods allow for the identification of novel therapeutics that modify the injury process, rather than merely mitigating the symptoms.

We have discovered that the progesterone receptor (PR) is expressed in a high percentage of human neurofibromas in a manner suggesting that the PR ligand, progesterone, may play an important role in tumor growth. The estrogen receptor (ER) has also been shown to be expressed in neurofibromas, albeit in a lower number of neurofibromas and demonstrates that a gonadotropic steroid receptor can be expressed in peripheral nerve sheath tumors.

Progesterone is a steroid hormone known primarily for its role in development and maintenance of the reproductive system. In females, plasma progesterone levels are low until the first ovulation occurs, approximately 6 to 9 months following menarche. Thereafter, plasma progesterone levels fluctuate from 0.3-3.0 nmol/L during the follicular phase of the menstrual cycle to 19.0-45.0 nmol/L during the luteal phase. The corpus luteum within the ovary is the major source of progesterone during the menstrual cycle and early in pregnancy. After 6-8 weeks of gestation, the placenta takes over as the major source of progesterone, and plasma progesterone levels climb to six times the levels during the luteal phase. These changes in plasma progesterone levels correlate well with the periods of rapid growth observed for neurofibromas. Males have low but detectable plasma progesterone levels. The major source of progesterone in males is the testis, where progesterone serves as an intermediate in the synthesis of testosterone.

Progesterone has been implicated in a wide range of biological processes outside of the reproductive tract. In the peripheral nervous system progesterone promotes myelination of regenerating nerves. Progesterone is synthesized by Schwann cells (the cell type which produces myelin), and PR has been detected in primary Schwann cell cultures from rats, suggesting the presence of an autocrine loop. Progesterone appears to promote myelin formation by binding to PR and stimulating transcription of the transcription factor Krox-20, which in turn stimulates transcription of several myelin protein genes. Thus, both local and systemic production of progesterone may contribute to neurofibroma growth.

Using immunofluoresence and histochemical techniques, we have determined that a large percentage of neurofibromas express the progesterone receptor (e.g., non-neoplastic tumor-associated cells). Given their location, neurofibromas likely cause nerve injury, which may lead to the recruitment of neighboring Schwann cells to help in repair. These neighboring Schwann cells may upregulate PR in an effort to promote remyelination. It has been proposed that Schwann cells involved in nerve repair dedifferentiate and lose mature Schwann cell markers, which could explain the absence of S-100 in the PR-expressing cells. Thus, based on our discovery, compounds that modulate the biological activity of gonatropic steroid receptors are useful for the treatment and prophylaxis of PNSTs.

If desired, a second therapeutic regimen may also be administered to the mammal being treated according to the present invention, including for example, tumor resection, radiotherapy, and chemotherapy. Optionally, the biological activity of more than one gonatropic receptor may be modulated, and preferably, the progesterone receptor is one of the receptors being modulated. The progesterone receptor is a member of the gonadotropic steroid receptor family encompassing the aforementioned progesterone receptor, estrogen receptor, and androgen receptor. Small molecule inhibitors to gonadotropic steroid receptors are well known in the art and include, without limitation, the estrogen receptor antagonists, tamoxifen, raloxifene, ICI164384 (N-n-butyl-N-methyl-11 -(3,17 beta-dihydroxyoestra-1,3,5(10)-trien-7 alpha-yl) undecamide), faslodex, idoxifene, toremifene, EM-652, TAS-108, and droloxifene; androgen receptor antagonists, bicalutamide, flutamide, and nilutamide; and aromatase inhibitors, atemestane, exemestane, letrozole, and anastrozole (F. J. Cummings, Clin Ther 24:C3-25, 2002; Sathya et al., Endocrinology, 143:3071-3082, 2002; Chwalisz et al., Ann NY Acad Sci 955:373-88, 2002; Elger et al., Steroids 65:713-723, 2000; Sanchez-Criado et al., J Endocrinol Invest, 23:151-157, 2000; Pooley et al., J Med Chem, 41:3461-3466, 1998).

Antiprogestins, such as mifepristone (RU486), are currently being used for the treatment of other hormonally responsive tumors which express PR, including breast carcinoma and meningiomas. Based on our discovery, antiprogestins may also be useful for the treatment of PNSTs as an alternative to surgery, to reduce the size of lesions so that smaller surgical procedures could be performed, and/or to slow malignant progression of plexiform neurofibromas. Other antiprogestins in development that can substituted for mifepristone include, but are not limited to, onapristone, lilipristone, ZK 137316, ZK 230 211, Org 31710, and Org 31806, the latter two developed by Organon, Inc. (West Orange, N.J.).

### EXAMPLE 1

### Screening for Progesterone Receptor Expression in Peripheral Nerve Sheath Tumors

Several subtypes of neurofibromas have been described. While morphologically similar, their clinicopathologic features differ considerably. Localized and diffuse cutaneous neurofibromas affect the dermis and subcutis. Since these proliferations are extraneural, the nerve of origin is difficult to identify. Only a minority, approximately 10%, of cutaneous neurofibromas are associated with NF-1. Localized intraneural and plexiform neurofibromas proliferate intraneurally. While localized intraneural neurofibromas affect a segment of nerve, plexiform neurofibromas involve either a plexus of nerves or multiple fascicles within a large nerve. Like the cutaneous subtypes, the majority of localized intraneural neurofibromas are sporadic. By contrast, plexiform neurofibromas are found almost exclusively in NF1 patients. Most NF1-associated neurofibromas appear around puberty and increase in number later in life, however plexiform neurofibromas present in early childhood and are thought to be congenital. Importantly, about 5% of plexiform neurofibromas undergo malignant progression to malignant peripheral nerve sheath tumors (MPNSTs). Massive soft tissue neurofibromas represent the rarest subtype and are restricted to NF1 patients. Microscopically, extraneural and plexiform components are usually present. Despite the enormous size of these lesions,.malignant progression is rare.

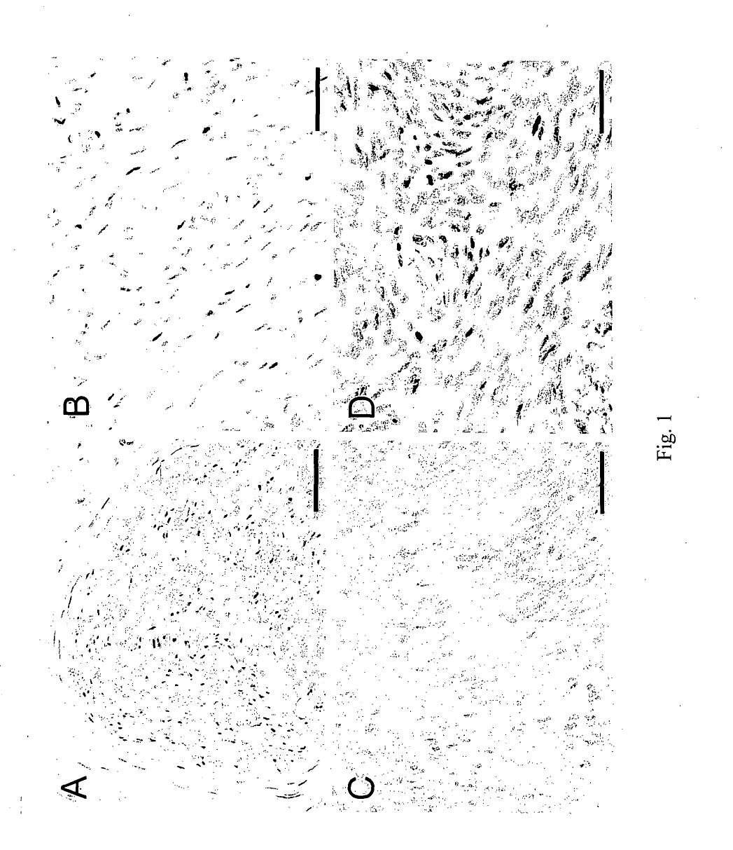

As a first step toward identifying the critical hormone(s) that regulates neurofibroma growth, we examined 59 human neurofibromas, including neurofibromas of each subtype, for the expression of estrogen receptor (ER) and PR. The majority (75%) of neurofibromas expressed PR (Table 1 and FIG. 1), while only a minority (5%) of neurofibromas expressed ER. PR expression was rarely seen in other peripheral nerve sheath tumors (schwannomas and MPNSTs) and was not detected in normal peripheral nerve. PR expression was found in every subtype of neurofibroma (Table 1).

TABLE 1
Progesterone Receptor expression in peripheral nerve sheath tumors

| | PR-positive tumors/total |
|---|---|
| Tumor Type | No. of tumors |
| Neurofibromas | 44/59 |
| Localized cutaneous neurofibromas | 17/20 |
| Diffuse cutaneous neurofibromas | 8/9 |
| Localized intraneural neurofibromas | 1/2 |
| Plexiform neurofibromas | 6/12 |
| Neurofibromas with extraneural and | 10/12 |
| plexiform features | |
| Deep-seated neurofibromas | 2/4 |
| Schwannomas | 1/7 |
| Malignant peripheral nerve sheath tumors | 1/10 |

### EXAMPLE 2

### Analysis of Progesterone Receptor Expression in Neurofibromas

The cutaneous neurofibroma subtypes were more frequently positive for PR (86%) than was the plexiform subtype (50%) (Table 2). This difference is statistically significant with a P value less than 0.025, and correlates with the clinical observation that the cutaneous subtypes first appear around the time of puberty while the plexiform subtype is thought to be congenital.

No significant difference was found between the frequency of PR-positive neurofibromas in males verse females (Table 2). When the plexiform neurofibromas which occur early in childhood in NF1 patients were excluded from the analysis, no significant difference was found between the frequency of PR-positive neurofibromas in patients less than or equal to 20 years of age and those over 20 years of age, and no significant difference was found between patients with NF1 and patients with sporadic neurofibromas (Table 2).

Eleven patients included in the study had multiple neurofibromas. In 10 out of 11 of these patients the pattern of PR expression in the neurofibromas was synchronized. In other words, if one neurofibroma was positive for PR then the other neurofibromas in the same patient were also positive for PR. One patient had 3 localized cutaneous neurofibromas which grew rapidly during pregnancy and were removed shortly after giving birth (at a time when progesterone levels would have decreased sharply since the placenta, the major source of the high levels of progesterone in pregnancy, was gone). All 3 neurofibromas in this patient were negative for PR. The finding of synchronized PR expression in multiple neurofibromas from individual patients suggests that systemic hormone levels may influence local PR expression within neurofibromas.

TABLE 2
Progesterone Receptor expression in subsets of neurofibromas

| | PR-positive tumors/total |
|---|---|
| Neurofibroma subset | No. of tumors |
| Localized and diffuse cutaneous neurofibromas | 25/29 |
| Plexiform neurofibromas | 6/12 |
| Males | 19/24 |
| Females | 25/35 |
| Patients less than or equal to 20 years of age* | 14/17 |
| Patients greater than 20 years of age* | 24/30 |
| NF1 patients | 19/29 |
| Sporadic neurofibromas | 14/18 |

*Plexiform neurofibromas excluded from the analysis

### EXAMPLE 3

### Analysis of Progesterone Receptor Isoform Expression in Neurofibromas

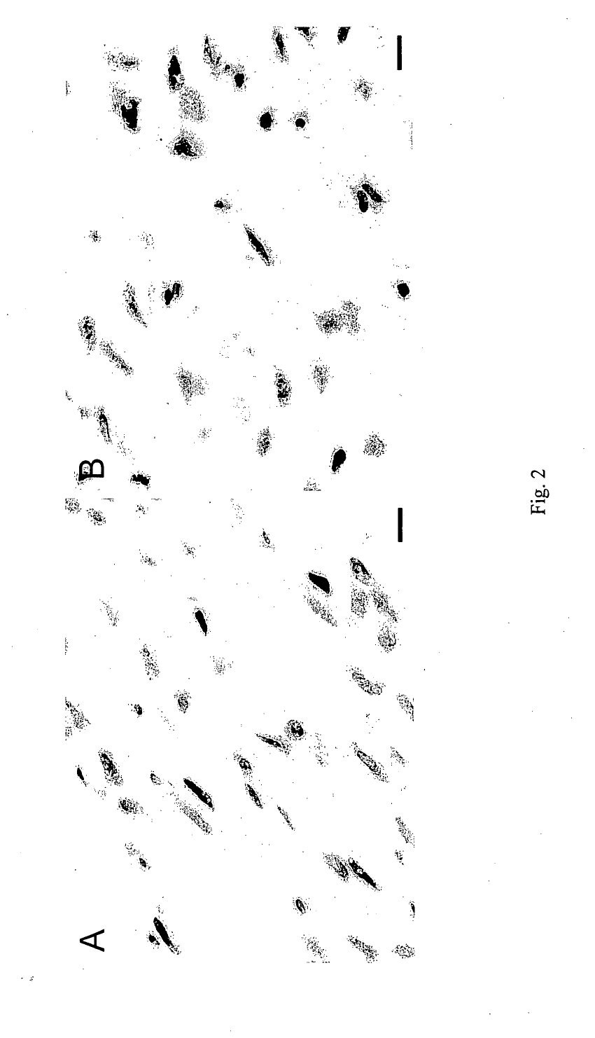

There are two isoforms of PR, PR-A and PR-B, which are transcribed from distinct estrogen-inducible promoters. In most contexts PR-B acts as a transcriptional activator, whereas PR-A acts as a transcriptional repressor. Both PR-A and PR-B contain an N-terminal inhibitory domain, however PR-B contains an extra 164 amino acid domain at the extreme N-terminus, which is thought to mask its inhibitory domain. PR-A is a transcriptional repressor of PR-B as well as of estrogen, glucocorticoid, androgen, and mineralcorticoid receptors. PR-A can heterodimerze with PR-B, and therefore may inhibit PR-B directly. PR-A, however, cannot heterodimerize with ER, suggesting that the mechanism of transcriptional interference is indirect and may involve binding to corepressors. In most PR-expressing cells, PR-A and PR-B are present in equimolar amounts. There are exceptions such as uterus, breast, and endometrial tumors which have been shown to have low PR-B expression levels. Since the pharmacological response to antiprogestins is likely determine by the relative expression of the two isoforms, we preformed immunohistochemistry on the human neurofibromas with antibodies specific for each isoform and detected strong expression of PR-A and PR-B (FIG. 2).

### EXAMPLE 4

### Analysis of Cell Type Expression of the Progesterone Receptor in Neurofibromas

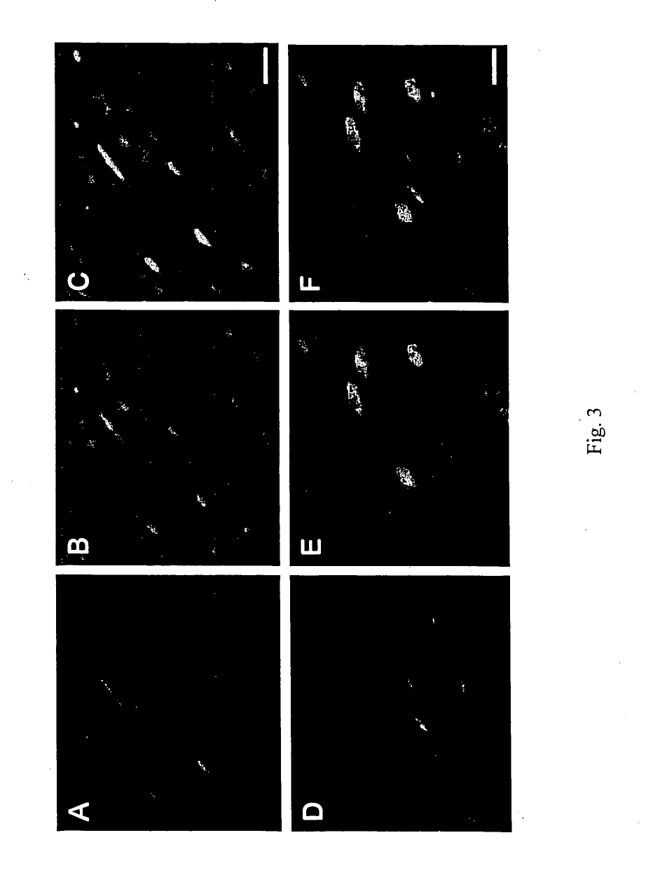

Within the PR-positive neurofibromas not every cell expresses PR. This is to be expected given the cellular heterogeneity of neurofibromas. To address the question of which cell type within the tumor expresses PR, we preformed double immunofluoresence on five neurofibromas from NF1 patients (FIG. 3). Neoplastic Schwann cells from these tumors should express the Schwann cell marker S-100, but lack the NF1 gene product neurofibromin. We found that in contrast to neoplastic Schwann cells, the PR-expressing cells contained neurofibromin and lacked S-100, indicating that they represent non-neoplastic tumor-associated cells.

### EXAMPLE 5

### Analysis of Estrogen Receptor Expression in Neurofibromas

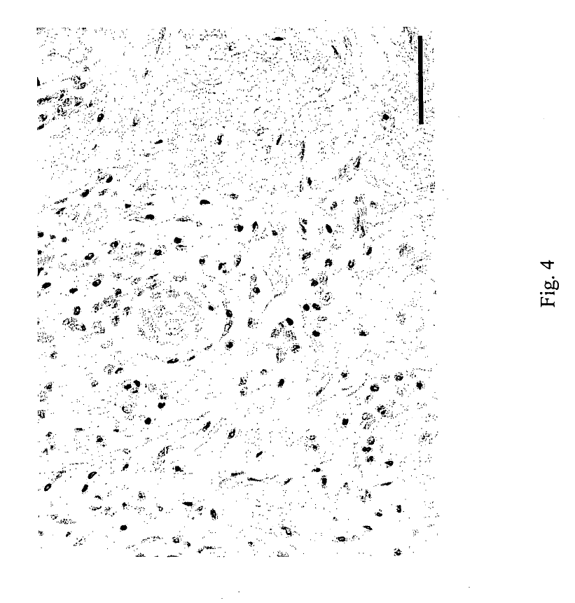

The estrogen receptor (ER) is expressed in a minority of neurofibromas. FIG. 4 is a photograph of an histological preparation of human peripheral nerve tissue and probed for estrogen receptor expression. Immunohistochemistry with an anti-ER antibody demonstrates that scattered cells within neurofibromas have nuclear staining for ER.

Materials and Methods

Human tissue sample acquisition. Formalin-fixed, paraffin-embedded tissue sections were obtained from 59 neurofibromas (20 localized cutaneous neurofibromas, 9 diffuse cutaneous neurofibromas, 2 localized intraneural neurofibromas, 12 plexiform neurofibromas, 12 mixed neurofibromas with features of both diffuse and plexiform neurofibromas, and 4 deep-seated neurofibromas), 7 schwannomas, 10 malignant peripheral nerve sheath tumors, and one normal peripheral nerve. Studies were done with the approval of the Committee for Clinical Investigation of Boston Children's Hospital and the Human Research Committee of Brigham and Women's Hospital.

Immunohistochemistry of human tissue samples. We stained formalin-fixed, paraffin-embedded tissue sections with (1) a 1:200 dilution of mouse monoclonal anti-human PR antibody (Clone PgR 636, DAKO; Carpinteria, Calif.), (2) a 1:200 dilution of mouse monoclonal anti-human ER antibody (Clone 1D5, DAKO), (3) a 1:100 dilution of mouse monoclonal anti-human PR antibody (NCL-L-PGR-312, Vector; Burlingame, Calif.), and (4) a 1:100 dilution of mouse monoclonal anti-human PR antibody (Clone hPRa 2, Lab Vision; Fremont, Calif.). We blocked endogenous peroxidases using 1% hydrogen peroxide in ethanol for (1) and (2), or 3% hydrogen peroxide in dH₂0 for (3) and (4). To unmask antigens we pretreated samples in 10 mM citrate buffer pH 6.0 for 30 min. in a microwave oven for (1) and (2), or in Triology buffer with EDTA (Cell Marque; Hot Springs, Ark.) for 15 min. in an electric pressure cooker for (3) and (4). We detected antibodies (1) and (2) with a secondary antibody conjugated to a peroxidase labeled polymer (EnVision+System, DAKO), using DAB (DAKO) as a substrate for peroxidase. We detected antibodies (3) and (4) with a biotin-conjugated secondary antibody and ABC (Vector), using DAB (Vector) as a substrate for peroxidase. Sections were counterstained with hematoxylin.

Immunofluoresence of human tissue samples. We stained formalin-fixed, paraffin-embedded tissue sections with (1) a 1:25 dilution of mouse monoclonal anti-human PR antibody (Clone PgR 636, DAKO), (2) a 1:100 dilution of rabbit polyclonal anti-human neurofibromin antibody (sc-67, Santa Cruz Biotechnology; Santa Cruz, Calif.), and (3) a 1:500 dilution of rabbit polyclonal anti-cow S-100 antibody (Z0311, DAKO). To unmask antigens we pretreated samples in Triology buffer with EDTA for 15 min in an electric pressure cooker. We detected antibody (1) with a rhodamine-conjugated anti-mouse secondary antibody at 1:200 dilution (Jackson ImmunoResearch; West Grove, Pa.), antibody (2) with a biotin-conjugated anti-rabbit secondary antibody at 1:1000 dilution (DAKO) and streptavidin-conjugated Oregon green at 1:300 dilution (Molecular Probes; Eugene, Oreg.), and antibody (3) with a FITC-conjugated anti-rabbit secondary antibody at 1:1000 dilution (Jackson ImmunoResearch). Sections were then counterstained for 5 min. with DAPI.

Therapeutic Uses

The present invention features methods for treating tumors of the neural sheath. Compounds of the present invention may be administered by any appropriate route for treatment or prevention of a disease or condition associated with angiogenesis associated diseases. These may be administered to any mammals including humans, domestic pets, livestock, or other animals with a pharmaceutically acceptable diluent, carrier, or excipient, in unit dosage form. Administration may be parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or oral administration.

Therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in “Remington: The Science and Practice of Pharmacy” (20th ed., ed. A. R. Gennaro, 2000, Lippincott Williams & Wilkins). Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Nanoparticulate formulations (e.g., biodegradable nanoparticles, solid lipid nanoparticles, liposomes) may be used to control the biodistribution of the compounds. Other potentially useful parenteral delivery systems include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycholate and deoxycholate, or may be oily solutions for administration in the form of nasal drops, or as a gel. The concentration of the compound in the formulation will vary depending upon a number of factors, including the dosage of the drug to be administered, and the route of administration.

The compound may be optionally administered as a pharmaceutically acceptable salt, such as a non-toxic acid addition salts or metal complexes that are commonly used in the pharmaceutical industry. Examples of acid addition salts include organic acids such as acetic, lactic, palmoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. Metal complexes include zinc, iron, and the like.

Administration of compounds in controlled release formulations is useful where the compound of formula I has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀)); (ii) a narrow absorption window in the gastro-intestinal tract; or (iii) a short biological half-life, so that frequent dosing during a day is required in order to sustain the plasma level at a therapeutic level.

Many strategies can be pursued to obtain controlled release in which the rate of release outweighs the rate of metabolism of the therapeutic compound. For example, controlled release can be obtained by the appropriate selection of formulation parameters and ingredients, including, e.g., appropriate controlled release compositions and coatings. Examples include single or multiple unit tablet or capsule compositions, oil solutions, suspensions, emulsions, microcapsules, microspheres, nanoparticles, patches, and liposomes.

Formulations for oral use include tablets containing the active ingredient(s) in a mixture with non-toxic pharmaceutically acceptable excipients. These excipients may be, for example, inert diluents or fillers (e.g., sucrose and sorbitol), lubricating agents, glidants, and antiadhesives (e.g., magnesium stearate, zinc stearate, stearic acid, silicas, hydrogenated vegetable oils, or talc)

Formulations for oral use may also be provided as chewable tablets, or as hard gelatin capsules wherein the active ingredient is mixed with an inert solid diluent, or as soft gelatin capsules wherein the active ingredient is mixed with water or an oil medium.

Gene Therapy

Gene therapy is another potential therapeutic approach in which copies of DNA encoding for the gonadotropic steroid receptors or fragments thereof are introduced into selected tissues to successfully encode for abundant polypeptide product in affected cell types (e.g., peripheral nerve sheath tumors). The DNA must be delivered to those cells in a form in which it can be taken up and encode for sufficient polypeptide product to provide effective function. Alternatively, genes encoding the gonadotropic steroid receptors or fragments thereof can be positioned in such a way as to produce antisense transcripts upon transcription.

Transducing retroviral vectors can be used for somatic cell gene therapy especially because of their high efficiency of infection and stable integration and expression. The full length DNA encoding gonadotropic steroid receptors, or portions thereof, can be cloned into a retroviral vector in either sense or antisense orientation, and driven from its endogenous promoter or from the retroviral long terminal repeat or from a promoter specific for the target cell type of interest (such as neurons). Other viral vectors that can be used include adeno-associated virus, vaccinia virus, bovine papilloma virus, or a herpes virus such as Epstein-Barr virus.

Gene transfer could also be achieved using non-viral means requiring infection of cancer cells in vitro. This would include calcium phosphate, DEAE dextran, electroporation, and protoplast fusion. Liposomes may also be potentially beneficial for delivery of DNA into a cell. Although these methods are available, many of these are lower efficiency.

Retroviral vectors, adenoviral vectors, adenovirus-associated viral vectors, or other viral vectors with the appropriate tropism for cells likely to be involved in diseases involving insufficient apoptosis may be used as a gene transfer delivery system for a therapeutic gonadotropic steroid receptor DNA construct. Numerous vectors useful for this purpose are generally known (Miller, Human Gene Therapy 15-14, 1990; Friedman, Science 244:1275-1281, 1989; Eglitis and Anderson, BioTechniques 6:608-614, 1988; Tolstoshev and Anderson, Curr. Opin. Biotech. 1:55-61, 1990; Sharp, The Lancet 337:1277-1278, 1991; Cometta et al., Nucl. Acid Res. and Mol. Biol. 36:311-322, 1987; Anderson, Science 226:401-409, 1984; Moen, Blood Cells 17:407-416, 1991; Miller et al., Biotech. 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

For any of the methods of application described above, the therapeutic gonadotropic steroid receptors-encoding DNA construct is preferably applied to the site of the desired therapeutic event (for example, by injection). However, it may also be applied to tissue in the vicinity of the desired therapeutic event or to a blood vessel supplying the cells (e.g., peripheral nerve sheath tumor cells).

In the constructs described, DNA expression can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in neural cells may be used to direct antisense messages of gonadotropic steroid receptors or fragments thereof.

### OTHER EMBODIMENTS

From the foregoing description, it is apparent that variations and modifications may be made to the invention described herein to adopt it to various usages and conditions. Such embodiments are also within the scope of the following claims.

All publications mentioned in this specification are herein incorporated by reference to the same extent as if each independent publication or patent application was specifically and individually indicated to be incorporated by reference.

1. A method of treating or preventing a peripheral nerve sheath tumor by administering to a mammal a therapeutically effective dose of a compound that modulates the biological activity of a gonadotropic steroid receptor.

2. The method of claim 1, wherein said peripheral nerve sheath tumor is prevented.

3. The method of claim 1, wherein said peripheral nerve sheath tumor is treated.

4. The method of claim 1, wherein said gonadotropic steroid receptor is a progesterone receptor.

5. The method of claim 1, wherein said gonatropic steroid receptor is an estrogen receptor.

6. The method of claim 1, wherein said gonatropic steroid receptor is an androgen receptor.

7. The method of claim 1, wherein said compound activates said biological activity of said gonatropic receptor.

8. The method of claim 1, wherein said compound inhibits said biological activity of said gonatropic receptor.

9. The method of claim 8, wherein said compound is selected from a group consisting of mifepristone, onapristone, lilipristone, Org 31710, Org 31806, tamoxifen, raloxifene, faslodex, TAS-108, droloxifen, ICI164384, atemestane, bicalutamide, flutamide, and nilutamide.

10. The method of claim 1, wherein said gonatropic steroid receptor is a progesterone receptor.

11. The method of claim 10, wherein said compound is selected from a group consisting of mifepristone, onapristone, lilipristone, Org 31710, and Org 31806.

12. The method of claim 11, wherein said compound is mifepristone.

13. The method of claim 1, wherein more than one gonatropic steroid receptor is modulated, said receptor selected from the group consisting of estrogen receptor, androgen receptor, and progesterone receptor.

14. The method of claim 13, wherein one of said gonatropic steroid receptor is the progesterone receptor.

15. The method of claim 1, further comprising a second therapeutic regimen.

16. The method of claim 15, wherein said second therapeutic regimen is tumor resection, chemotherapy, or radiotherapy.

17. The method of claim 1, wherein said compound is a small molecule antagonist, a neutralizing antibody, an antisense nucleic acid, or a double stranded interference ribonucleic acid (RNAi).

18. The method of claim 1, wherein said peripheral nerve sheath tumor is selected from a group consisting of neurofibromas, schwannomas, perineuriomas, malignant peripheral nerve sheath tumors, and Triton tumors.

19. The method of claim 18, wherein said peripheral nerve sheath tumor is a neurofibroma.

20. The method of claim 19, wherein said neurofibroma is a sporadic neurofibroma.

21. The method of claim 19, wherein said neurofibroma is associated with type-1 neurofibromatosis.

22. A method of monitoring the progression of a peripheral nerve sheath tumor, said method comprising measuring the amount of a gonadotropic steroid receptor mRNA or polypeptide expression in a sample from a mammal, wherein an increase or decrease in said gonadotropic steroid receptor MRNA or polypeptide expression in said sample relative to a control sample indicates a progression of a peripheral nerve sheath tumor or a propensity thereto in said mammal.

23. A method of determining a course of treatment of a mammal diagnosed as having a peripheral nerve sheath tumor, said method comprising the steps of:
a) providing a histological preparation of a peripheral nerve sheath tumor from said mammal; and
b) detecting the presence of a gonadotropic steroid receptor in said histological preparation, wherein the presence of said gonadotropic steroid receptor identifies said mammal as being a candidate for treatment with a compound that modulates the biological activity of a gonadotropic steroid receptor.

24. The method of claim 23, wherein said compound inhibits said gonatropic steroid receptor.

25. The method of claim 23, wherein said compound activates said gonatropic steroid receptor.

26. The method of claim 23, wherein said gonadotropic steroid receptor is the progesterone receptor.

27. A method for identifying a candidate compound for treating, reducing, or preventing a peripheral nerve sheath tumor in a mammal, said method comprising:
(a) contacting a cell expressing a gonatropic receptor gene with a candidate compound; and
(b) measuring the gene expression or protein activity of said gonatropic receptor in said cell, a candidate compound that modulates said expression or said activity, relative to expression or activity of said gonatropic receptor in a cell not contacted with said candidate compound, identifying said candidate compound as a candidate compound useful for treating or preventing a peripheral nerve sheath tumor in a mammal.
